(19) United States
(12) Patent Application Publication
(10) Pub. No.: US 2022/0305190 A1
(43) Pub. Date: Sep. 29, 2022

(54) SUCTION AND IRRIGATION APPARATUS

(71) Applicant: ORANGE MEDICS, INC., Daejeon (KR)

(72) Inventor: Ki Hwan LEE, Daejeon (KR)

(73) Assignee: ORANGE MEDICS, INC., Daejeon (KR)

(21) Appl. No.: 17/431,716

(22) PCT Filed: Sep. 29, 2020
(86) PCT No.: PCT/KR2020/013342

§ 371 Date: Aug. 18, 2021

(30) Foreign Application Priority Data
Feb. 24, 2020 (KR) 10-2020-0022024

(51) Int. Cl. A61M 1/00; A61B 18/14

(52) U.S. Cl. CPC A61M 1/774; A61B 18/1482; A61B 2018/00589

(57) ABSTRACT

A suction and irrigation apparatus includes: a handle having a mounting space therein; an insertion unit extending from the handle and inserted into a patient's abdominal cavity; and a suction and supply unit disposed in the mounting space and supplying an irrigation fluid to the insertion unit or suctioning blood or other contaminated fluids from the patient's abdominal cavity through the insertion unit.

### TECHNICAL FIELD

The present invention relates to a suction and irrigation apparatus, and more particularly to a suction and irrigation apparatus which is easy to operate.

### BACKGROUND ART

In general, endoscopic (or laparoscopic) surgery is minimally invasive surgery and has been rapidly developing in recent years due to advantages thereof, such as less pain, less infection, minimal incision, and reduced hospital stay.

Laparoscopic surgery uses a surgical instrument for supplying an irrigation solution for irrigation of a surgical site of a patient and removing contaminated fluids after irrigation of the surgical site through discharge of the contaminated fluids from the patient's body, that is, a laparoscopic suction and irrigation system.

A typical laparoscopic suction and irrigation system includes a cannula inserted into a patient's body, a main body having a mixing chamber connected to the cannula, an irrigation path connected to the mixing chamber to supply an irrigation fluid into the patient's body therethrough, and a discharge path adapted to discharge contaminated fluids from the patient's body therethrough.

Recently, a component for an electrosurgical operation to create an incision at a surgical site is further incorporated into such a laparoscopic suction and irrigation system.

In general, in laparoscopic surgery using the laparoscopic suction and irrigation system, an electrosurgical operation, supply of an irrigation fluid, and removal of contaminated fluids are rapidly performed. Therefore, there is a need for a laparoscopic suction and irrigation system which is easy to operate and thus can ensure improved surgical efficiency.

As an example of prior documents, there is Korean Patent Registration No. 1837055 (published on Mar. 9, 2018).

### DISCLOSURE

### Technical Problem

Embodiments of the present invention are conceived to solve such problems in the art and provide a suction and irrigation apparatus which is easy to operate.

It will be understood that aspects of the present invention are not limited to the above one. The above and other aspects of the present invention will become apparent to those skilled in the art from the detailed description of the following embodiments in conjunction with the accompanying drawings.

### Technical Solution

In accordance with one aspect of the present invention, there is provided a suction and irrigation apparatus including: a handle having a mounting space therein; an insertion unit extending from the handle and inserted into a patient's abdominal cavity; and a suction and supply unit disposed in the mounting space and supplying an irrigation fluid to the insertion unit or suctioning blood or other contaminated fluids from the patient's abdominal cavity through the insertion unit.

The suction and supply unit may have: a housing having a first chamber and a second chamber separated from each other by an inner partition, a first connector and a second connector connected to the first chamber, and a third connector connected to the second chamber; a first valve provided to the first chamber, allowing a first flow path space to be defined between the first valve and the first chamber, and establishing selective connection between the first connector, the first flow path space, and the second connector while reciprocating in an axial direction of the first chamber; and a second valve provided to the second chamber, allowing a second flow path space to be defined between the second valve and the second chamber, and establishing selective connection between the second flow path space and the third connector while reciprocating in an axial direction of the second chamber.

The first chamber and the second chamber may be connected to each other through a connection hole formed through the inner partition.

The first valve may have a first valve body spaced apart from an inner peripheral surface of the first chamber, a first flange protruding from one end of the first valve body and closely contacting the inner peripheral surface of the first chamber, and a second flange protruding from the other end of the first valve body and closely contacting the inner peripheral surface of the first chamber.

Here, the first flow path space may be defined between the first valve body and the inner peripheral surface of the first chamber to be moved in conjunction with movement of the first valve.

The first valve may further have a first sealing ring disposed on an outer peripheral surface of the first flange and a second sealing ring disposed on an outer peripheral surface of the second flange.

The first valve body may have a first insertion groove axially formed at one end thereof, and the suction and supply unit may further have a first resilient member disposed inside the first chamber and having one end supported by the first chamber and the other end inserted into the first insertion groove to resiliently support the first valve.

The first valve body may further have a first support protrusion formed on the first insertion groove in the axial direction of the first valve body and disposed inside the first resilient member.

The first chamber may have a first securing protrusion surrounding one end of the first resilient member.

The connection hole may allow the first flow path space and the second flow path space to be constantly connected to each other regardless of the position of the first valve and the second valve.

The insertion unit may have: an electrically conductive cannula rotatably coupled at one end thereof to the first connector, extending from the handle, and coupled to an electrode; an adapter rotatably mounted on the handle and coupled to the cannula to be rotatable in conjunction with rotation of the cannula; an insulating protective tube covering the cannula; and an adjuster mounted on the handle and selectively coupled to the adapter while reciprocating in a longitudinal direction of the cannula.

The adapter may have a coupling protrusion protruding toward the adjuster, and the adjuster may have a coupling groove receiving the coupling protrusion therein.

When the adjuster is rotated with the coupling protrusion received in the coupling groove, the adapter may be rotated due to friction between the coupling protrusion and the coupling groove.

The insertion unit may further have: a clamp coupled to one end of the first connector and having a third through-hole through which the cannula passes; and a sealing member disposed between the clamp and the first connector and allowing the cannula to pass therethrough.

### Advantageous Effects

According to embodiments of the present invention, the adjuster can be selectively coupled to the adapter while freely reciprocating in the longitudinal direction of the cannula, whereby an operation of exposing or rotating the electrode can be performed simply through manipulation of the adjuster, thereby ensuring easy operation.

In addition, according to the embodiments of the present invention, the suction and supply unit 300 can perform supply of an irrigation fluid, suction of blood or other contaminated fluids from a patient's abdominal cavity, or irrigation of the second connector, the first tube, and the interiors of the first chamber and the second chamber simply through manipulation of the first valve and the second valve, thereby improving user convenience.

It will be understood that advantageous effects of the present invention are not limited to the above ones, and include any advantageous effects conceivable from the features disclosed in the detailed description of the present invention or the appended claims.

### DESCRIPTION OF DRAWINGS

FIG. 1 is a perspective view of a suction and irrigation apparatus according to an embodiment of the present invention.

FIG. 2 is a sectional view of the suction and irrigation apparatus of FIG. 1.

FIG. 3 is a sectional view of the suction and irrigation apparatus, focused on a projection of a handle shown in FIG. 2.

FIG. 4 is an exploded perspective view of the suction and irrigation apparatus of FIG. 1, focused on an insertion unit thereof.

FIG. 5 is a sectional view of the suction and irrigation apparatus of FIG. 1, illustrating operation of the insertion unit.

FIG. 6 is a perspective view of a suction and supply unit of the suction and irrigation apparatus of FIG. 1.

FIG. 7 is a sectional view taken along line A-A′ of FIG. 6.

FIG. 8 is a sectional view taken along line B-B′ of FIG. 6.

FIG. 9 is a sectional view taken along line C-C′ of FIG. 6.

FIG. 10 is a sectional view of the suction and irrigation apparatus of FIG. 1, focused on an electricity supply unit thereof.

### BEST MODE

Reference will now be made in detail to various embodiments, examples of which are illustrated in the accompanying drawings. It should be understood that the present invention may be embodied in different ways and is not limited to the following embodiments. In the drawings, portions irrelevant to the description will be omitted for clarity. Like components will be denoted by like reference numerals throughout the specification.

Throughout the specification, when an element or layer is referred to as being “on,” “connected to,” or “coupled to” another element or layer, it may be directly on, connected to, or coupled to the other element or layer or intervening elements or layers may be present. In addition, unless stated otherwise, the term “includes” should be interpreted as not excluding the presence of other components than those listed herein.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Hereinafter, embodiments of the present invention will be described with reference to the accompanying drawings.

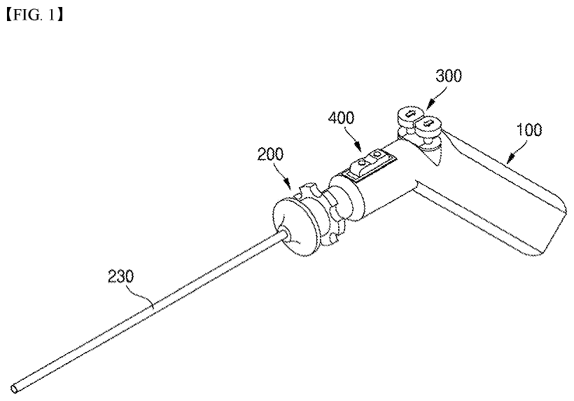

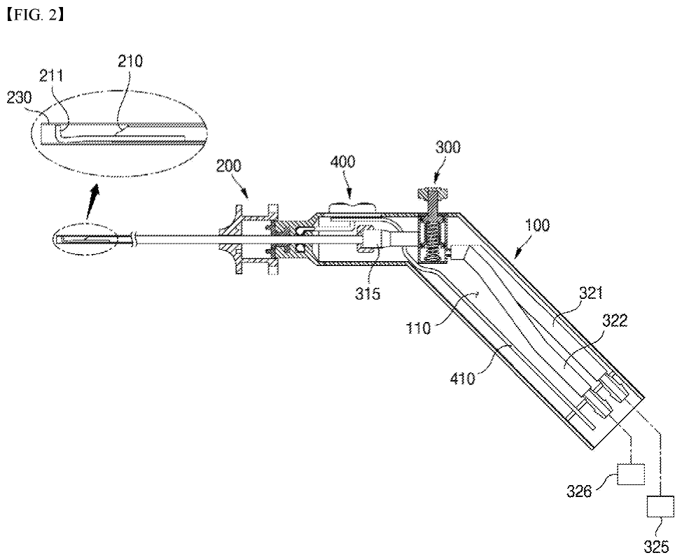

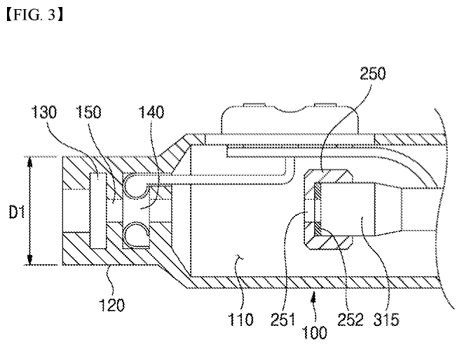

FIG. 1 is a perspective view of a suction and irrigation apparatus according to one embodiment of the present invention, FIG. 2 is a sectional view of the suction and irrigation apparatus of FIG. 1, and FIG. 3 is a sectional view of the suction and irrigation apparatus, focused on a projection of a handle shown in FIG. 2.

Referring to FIG. 1 to FIG. 3, the suction and irrigation apparatus includes a handle, 100, an insertion unit 200, and a suction and supply unit 300.

The handle 100 may have a mounting space 110 therein.

The insertion unit 200 may extend from the handle 100 and may be inserted into a patient's abdominal cavity.

The suction and supply unit 300 may be disposed in the mounting space 110 to supply an irrigation fluid to the insertion unit 200 or to suction blood or other contaminated fluids from the patient's abdominal cavity through the insertion unit 200.

Specifically, the handle 100 may be grasped by a hand of a user and may have the mounting space 110 therein. In addition, the handle 100 may have a projection 120 formed at one end thereof. The projection 120 may have a first diameter D1. Further, the handle 100 may have a first mounting groove 130, a second mounting groove 140, and a connection groove 150.

The first mounting groove 130 and the second mounting groove 140 may be formed in the projection 120, may be coaxial with the projection 120, and may be formed in a circumferential direction of the projection 120. The connection groove 150 may be axially formed in the projection 120 and may pass through the first mounting groove 130 and the second mounting groove 140.

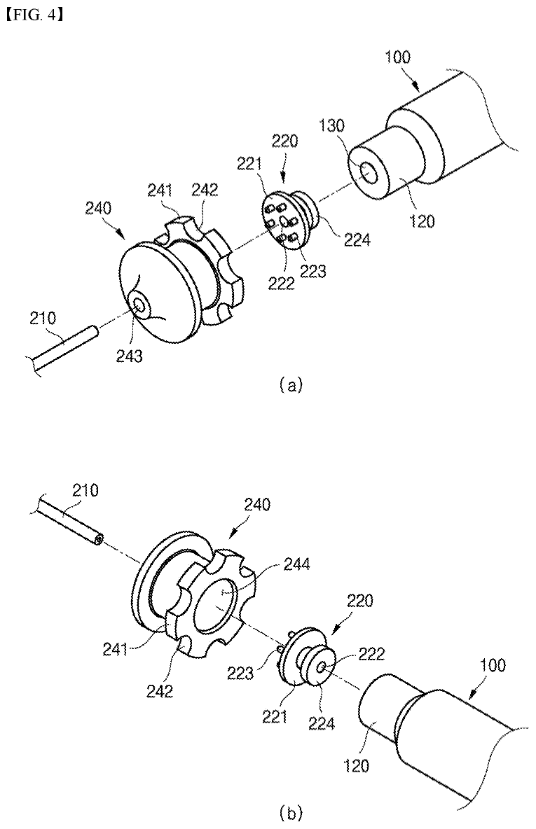

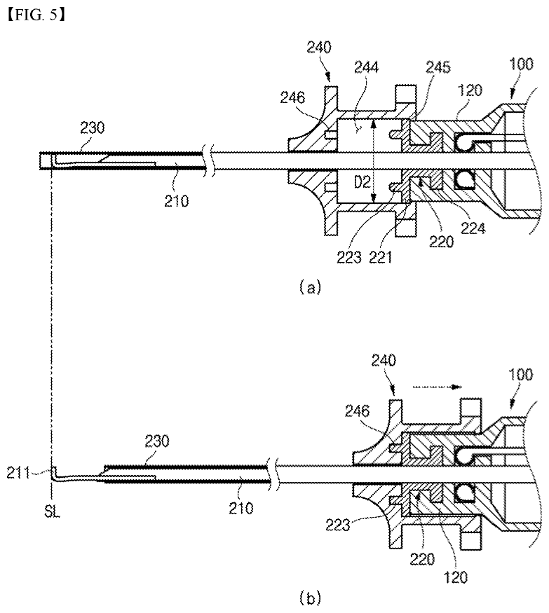

FIG. 4 is an exploded perspective view of the suction and irrigation apparatus of FIG. 1, focused on the insertion unit, and FIG. 5 is a sectional view of the suction and irrigation apparatus of FIG. 1, illustrating operation of the insertion unit.

Referring to FIG. 4 and FIG. 5 in conjunction with FIG. 3, the insertion unit 200 may have a cannula, an adapter 220, a protective tube 230, and an adjuster 240.

The cannula 210 may have one end inserted into the connection groove 150 of the handle 100. The one end of the cannula 210 may extend to the mounting space 110 through the second mounting groove 140 and may be rotatably coupled to a first connector 315 described below. In addition, the cannula 210 may extend from the handle 100 to be inserted into the patient's abdominal cavity. The cannula 210 may have electrical conductivity.

The cannula 210 may have the other end coupled to an electrode 211. The electrode 211 may be heated by electricity delivered through the cannula 210. Electricity may be selectively supplied to the cannula 210 such that the electrode 211 can be selectively heated. The electrode 211 may be used to perform an electrosurgical operation, such as creating an incision at a patient's surgical site and causing coagulation at the patient's surgical site.

The adapter 220 may be rotatably mounted on the handle 100. The adapter 220 may have a first locking plate 221 and a second locking plate 224.

The first locking plate 221 may closely contact one end of the projection 120 of the handle 100. The first locking plate 221 may have a larger diameter than the first diameter D1 of the projection 120. Accordingly, when the first locking plate 221 closely contacts the one end of the projection 120, the first locking plate 221 may be stepped with respect to the projection 120.

The first locking plate 221 may have a coupling protrusion 223 extending from one surface thereof toward a coupling groove 246, that is, toward the adjuster. The coupling protrusion 223 may include multiple coupling protrusions 223 arranged in a circumferential direction of the first locking plate 221. However, it will be understood that the present invention is not limited thereto and the coupling protrusion 223 may be formed in a continuous annular shape in the circumferential direction of the first locking plate 221.

The second locking plate 224 may be inserted into the first mounting groove 130 of the projection 120. The second locking plate 224 may be rotatably received in the first mounting groove 130.

The adapter 220 may have a first through-hole 222 along a central axis thereof. The first through-hole 222 is adapted to for the cannula 210 to be inserted thereinto. In one embodiment, the cannula 210 may be fitted into the first through hole 222. Accordingly, the cannula 210 may be rotated in conjunction with rotation of the adapter 220.

However, it will be understood that the present invention is not limited thereto. In another embodiment, the first through hole 222 may have a guide groove formed on an inner peripheral surface thereof and the cannula 210 may have an insertion protrusion formed on an outer surface thereof and inserted into the guide groove to allow the cannula 210 to be rotated in conjunction with rotation of the adapter 220.

The protective tube 230 may cover the cannula 210. The protective tube 230 may receive the cannula 210 therein to protect the cannula 210. With the cannula 210 in a stationary position, the protective tube 230 may be rotatable or movable in a longitudinal direction of the cannula 210. The protective tube 230 may be formed of an insulating material.

The adjuster 240 may have a second through-hole 243 axially formed therethrough and a receiving groove 244.

The second through-hole 243 allows the protective tube 230 to be inserted thereinto. The protective tube 230 may be fitted into the second through-hole 243. Accordingly, the protective tube 230 may be rotated in conjunction with rotation of the adjuster 240.

The receiving groove 244 may have a second diameter D2 larger than the first diameter D1 of the projection 120 and may receive the first locking plate 221 of the adapter 220 therein. When the adjuster 240 is coupled to the projection 120 of the handle 100 such that the first locking plate 221 is received in the receiving groove 244 with the adapter 220 coupled to the projection 120, the adjuster 240 may be forced to reciprocate in a longitudinal direction of the projection 120.

The adjuster 240 may have a locking stepped portion 245 formed at one end thereof. The locking stepped portion 245 may have a smaller diameter than the second diameter D2 of the receiving groove 244. Preferably, the locking stepped portion 245 has a smaller diameter than an outer diameter of the first locking plate 221 of the adapter 220. When the adjuster 240 is in a position as shown in FIG. 5(a), the locking stepped portion 245 may be caught by the first locking plate 221, thereby preventing the adjuster 240 from being separated from the adapter 220.

Here, each of the handle 100 and the adjuster 240 may be assembled by coupling two half-housings to each other.

The adjuster 240 may have a coupling groove 246 formed inside the receiving groove 244. The coupling groove 246 may be formed in the circumferential direction of the adjuster 240 to receive the coupling protrusion 223 therein. When the adjuster 240 is moved toward the adapter 220 with one end of the electrode 211 lying on the same line SL, as shown in FIG. 5(b), the protective tube 230 may be moved in conjunction with the adjuster 240 such that the electrode 211 can be exposed to an outside environment.

In addition, the coupling protrusion 223 may be coupled to the coupling groove 246. Here, the coupling protrusion 223 may be fitted into the coupling groove 246. When the adjuster 240 is rotated with the coupling protrusion 223 fitted in the coupling groove 246, torque of the adjuster 240 may be transmitted to the adapter 220 through the coupling protrusion 223 fitted in the coupling groove 246. That is, when the adjuster 240 is rotated with the coupling protrusion 223 fitted in the coupling groove 246, the adapter 240 is also rotated due to friction between the coupling protrusion 223 and the coupling groove 246.

When the adapter 220 is rotated, the cannula 210 fitted in the adapter 220 is rotated and thus the electrode 211 coupled to the cannula 210 is also rotated. In addition, when the adjuster 240 is rotated with the coupling protrusion 223 fitted in the coupling groove 246, the protective tube 230 coupled to the adjuster 240 is also rotated.

Since the coupling groove 246 is continuously formed in the circumferential direction of the adjuster 240, the coupling protrusion 223 can be inserted into the coupling groove 246 regardless of rotational position of the adjuster 240.

When the adjuster 240 is rotated with the coupling groove 246 disengaged from the coupling protrusion 223, the adapter 220 is not rotated and only the protective tube 230 is rotated.

Accordingly, whether the electrode 211 is rotated upon rotation of the adjuster 240 depends on whether the coupling groove 246 is coupled to the coupling protrusion 223. That is, the electrode 211 is only rotated after the electrode 211 is exposed and the coupling protrusion 223 is fitted into the coupling groove 246 through movement of the adjuster 240 in the longitudinal direction of the cannula 210.

As such, fitting engagement between the coupling protrusion 223 and the coupling groove 246 can prevent the adjuster 240 from being easily moved in the longitudinal direction of the cannula 210 during an electrosurgical operation performed by an operator using the electrode 211.

Since the adjuster 240 can be selectively coupled to the adapter 220 while being forced to reciprocate in the longitudinal direction of the cannula 210 by external force applied by an operator, an operation of exposing or rotating the electrode 211 can be performed simply through manipulation of the adjuster 240, thereby ensuring easy operation. Further, the adjuster 240 may have a knob 241. The knob 241 may have recesses 242 on an outer surface thereof such that a user can easily rotate the knob 241 without slipping.

In addition, the insertion unit 200 may have a clamp 250 and a sealing member 252.

The clamp 250 may be coupled to one end of the first connector 315 and may have a third through-hole 251 through which the cannula 210 passes. That is, the cannula 210 may be inserted into the first connector 315 through the third through-hole 251 of the clamp 250.

The sealing member 252 may be disposed between the clamp 250 and the first connector 315, and the cannula 210 may pass through the sealing member 252. The sealing member 252 seals a gap between the clamp 250 and the first connector 315 to prevent leakage of an irrigation solution, blood, or other contaminated fluids moved through the first connector 315 and the cannula 210.

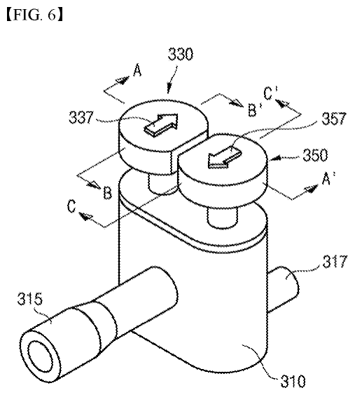

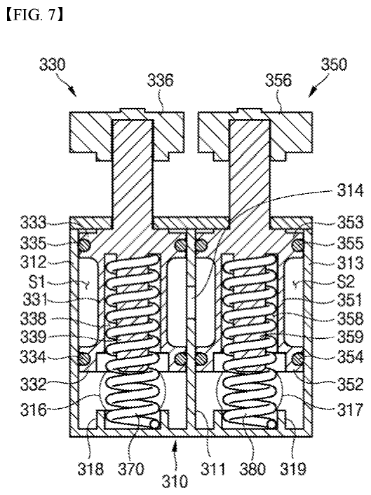

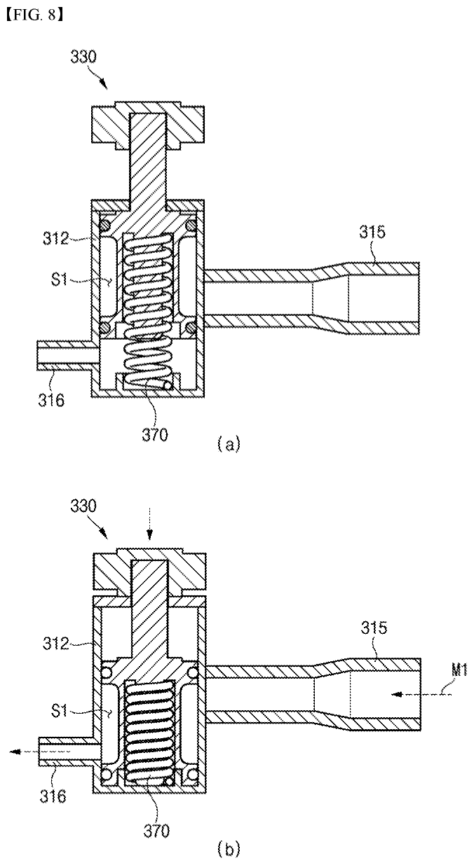

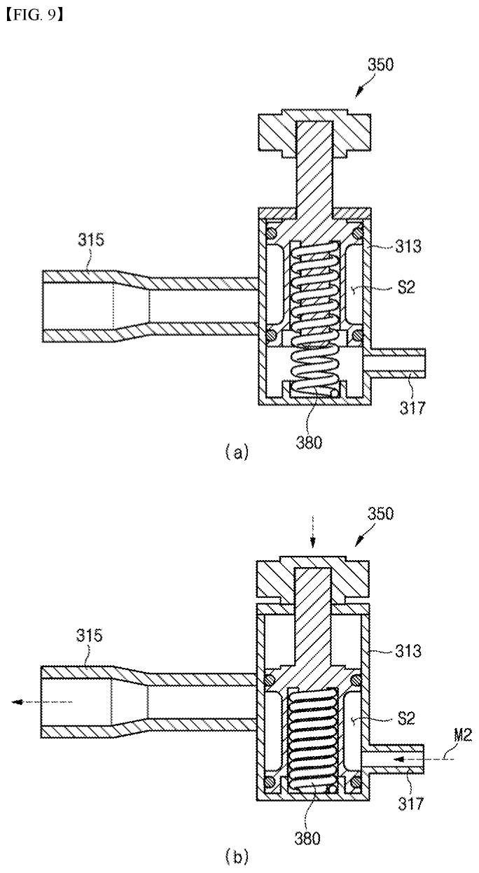

FIG. 6 is a perspective view of the suction and supply unit of the suction and irrigation apparatus of FIG. 1, FIG. 7 is a sectional view taken along line A-A′ of FIG. 6, and FIG. 8 is a sectional view taken along line B-B′ of FIG. 6, and FIG. 9 is a sectional view taken along line C-C′ of FIG. 6.

Referring to FIG. 6 to FIG. 9, the suction and supply unit 300 may have a housing 310, a first valve 330, and a second valve 350.

The housing 310 may have: an inner partition 311; and a first chamber 312 and a second chamber 313 separated from each other by the inner partition 311. The inner partition 311 may have a connection hole 314 formed therethrough. The first chamber 312 may be connected to the second chamber 313 through the connection hole 314.

In addition, the housing 310 may have a first connector 315, a second connector 316, and a third connector 317.

The first connector 315 may be connected to the first chamber 312 at one side of the housing 310. The second connector 316 may be connected to the first chamber 312 at the other side of the housing 310. The second connector 316 may be formed at a lower position than the first connector 315. The third connector 317 may be connected to the second chamber 313 at the other side of the housing 310. The third connector 317 may also be formed at a lower position than the first connector 315.

The first valve 330 may be provided to the first chamber 312. The first valve 330 may have a first valve body 331, a first flange 332, and a second flange 333.

The first valve body 331 may be disposed inside the first chamber 312 and may be forced to reciprocate in an axial direction of the first chamber 312. In addition, an outer peripheral surface of the first valve body 331 may be spaced apart from an inner peripheral surface of the first chamber 312. Accordingly, a first flow path space S1 may be formed between the first valve body 331 and the first chamber 312.

The first flange 332 may protrude from one end of the first valve body 331 and may closely contact the inner peripheral surface of the first chamber 312. In addition, the second flange 333 may protrude from the other end of the first valve body 331 and may closely contact the inner peripheral surface of the first chamber 312. That is, the first flow path space S1 may be defined by the first flange 332, the second flange 333, the first valve body 331, and the first chamber 312. When the first valve 330 is moved, the first flow path space S1 may also be moved.

In order to improve sealing at the first flange 332 and the second flange 333, the first valve 330 may have a first sealing ring 334 disposed on an outer peripheral surface of the first flange 332 and a second sealing ring 335 disposed on an outer peripheral surface of the second flange 333.

The first valve body 331 may have a first insertion groove 338 axially formed at one end thereof.

In addition, the suction and supply unit 300 may have a first resilient member 370. The first resilient member 370 may be disposed inside the first chamber 312 and may have one end supported by the first chamber 312 and the other end inserted into the first insertion groove 338 to resiliently support the first valve 330. The first valve body 331 may further have a first support protrusion 339 formed on the first insertion groove 338 in the axial direction of the first valve body 331 and disposed inside the first resilient member 370.

For convenience of description, with reference to FIG. 7, a moving direction of the first valve 330 causing compression of the first resilient member 370 will be referred to as “downward direction” and a moving direction of the first valve 330 causing extension of the first resilient member 370 will be referred to as “upward direction”.

The first chamber 312 of the suction and supply unit 300 may have a first securing protrusion 318 formed on a bottom thereof and surrounding a lower end of the first resilient member 370 to stably support both ends of the first resilient member 370.

The first valve 330 may further have a first push portion 336 formed at an upper end of the first valve body 331 and a first indicator 337 formed on an upper surface of the first push portion 336. The first indicator 337 may indicate a fluid flow direction upon downward movement of the first valve 330 by depression of the first push portion 336.

The first flow path space S1 may constantly be connected to the first connector 315 and the connection hole 314 formed through the inner partition 311 regardless of the position of the first valve 330.

The first valve 330 may establish selective connection between the first connector 315, the first flow path space S1, and the second connector 316 while reciprocating in the axial direction of the first chamber 312. Specifically, when the first valve 330 is pushed upward by elastic restoring force of the first resilient member 370 to be positioned above the first chamber 312, the first flow path space S1 may be connected to the first connector 315 without being connected to the second connector 316.

The second valve 350 may be provided to the second chamber 313. The second valve 350 may have a second valve body 351, a third flange 352, and a fourth flange 353.

The second valve body 351 may be disposed inside the second chamber 313 and may be forced to reciprocate in an axial direction of the second chamber 313. In addition, an outer peripheral surface of the second valve body 351 may be spaced apart from an inner peripheral surface of the second chamber 313. Accordingly, a second flow path space S2 may be formed between the second valve body 351 and the second chamber 313.

The third flange 352 may protrude from one end of the second valve body 351 and may closely contact the inner peripheral surface of the second chamber 313. In addition, the fourth flange 353 may protrude from the other end of the second valve body 351 and may closely contact the inner peripheral surface of the second chamber 313. That is, the second flow path space S2 may be defined by the third flange 352, the fourth flange 353, the second valve body 351, and the second chamber 313. When the second valve 350 is moved, the second flow path space S2 may also be moved.

In order to improve sealing at the third flange 352 and the fourth flange 353, the second valve 350 may have a third sealing ring 354 disposed on an outer peripheral surface of the third flange 352 and a fourth sealing ring 355 disposed on an outer peripheral surface of the fourth flange 353.

In addition, the suction and supply unit 300 may have a second resilient member 380. The second resilient member 380 may be disposed inside the second chamber 313 and may have one end supported by the second chamber 313 and the other end supported by the second valve 350 to resiliently support the second valve 350.

The second chamber 313 may have a second securing protrusion 319 formed on a bottom thereof and surrounding a lower end of the second resilient member 380, and the second valve body 351 may have a second insertion groove 358 formed in the axial direction thereof and receiving an upper end of the second resilient member 380 therein.

The second valve body 351 may further have a second support protrusion 359 formed on the second insertion groove 358 in the axial direction of the second valve body 351 and disposed inside the second resilient member 380.

The second valve 350 may further have a second push portion 356 formed at an upper end of the second valve body 351 and a second indicator 357 formed on an upper surface of the first push portion 356. The second indicator 357 may indicate a fluid flow direction upon downward movement of the second valve 350 by depression of the second push portion 356.

The second flow path space S2 may constantly be connected to the connection hole 314 formed through the inner partition 311 regardless of the position of the second valve 350.

The second valve 350 may establish selective connection between the second flow path space S2 and the third connector 317 while reciprocating in the axial direction of the second chamber 313. Specifically, when the second valve 350 is pushed upward, the second flow path space S2 may be disconnected from the third connector 317. When the second valve 350 is moved downward, the second flow path space S2 may be connected to the third connector 317. Except for the first connector 315, the suction and supply unit 300 may have a generally symmetrical shape with respect to the inner partition 311.

Referring to FIG. 2, the second connector 316 may be connected to a suction unit 325 through a first tube 321 and the third connector 317 may be connected to a pump 326 through a second tube 322. The pump 326 may supply an irrigation fluid.

When the first valve 330 is moved upward, as shown in FIG. 8(a), suction force applied by the suction unit 325 is not delivered to the first flow path space S1 despite being delivered to the second connector 316 and thus blood or contaminated fluids cannot be drawn from a patient's abdominal cavity into the first flow path space S1.

On the contrary, when the first valve 330 is moved downward by depression of the first push portion 336 by a user, as shown in FIG. 8(b), the first flow path space S1 is connected to both the second connector 316 and the first connector 315. As a result, suction force applied by the suction unit 325 can be delivered to the cannula 210 through the first connector 315, whereby blood or contaminated fluids M1 can be drawn from the patient's abdominal cavity into the first flow path space S1. Here, suction force applied by the suction unit 325 allows the blood or contaminated fluids M1 in the first flow path space S1 to be moved only to the second connector 316 without being moved to the second flow path space S2 through the connection hole 314 of the inner partition 311.

When the second valve 350 is in an upward position, as shown in FIG. 9(a), the irrigation fluid from the pump 326 is not delivered to the second flow path space S2 despite being delivered to the third connector 317. Thus, the irrigation fluid cannot be delivered to the patient's abdominal cavity.

On the contrary, when the second valve 350 is moved downward by depression of the second push portion 356 by a user, as shown in FIG. 9(b), the second flow path space S2 is connected to the third connector 317. Since the connection hole 314 is constantly connected to the first flow path space S1 and the second flow path space S2 regardless of position of the first valve 330 and the second valve 350, as described above, the irrigation fluid M2 moved to the second flow path space S2 can be moved to the first flow path space S1 through the connection hole 314. Here, when the first valve 330 is in an upward position in which the first flow path space S1 is connected only to the first connector 315, the irrigation fluid M2 in the first flow path space S1 can be moved to the first connector 315 to be delivered to a patient through the cannula 210. On the other hand, when both the second valve 350 and the first valve 330 are in a downward position, the irrigation fluid M2 in the first flow path space S1 can be delivered to the suction unit 325 through the second connector 316 by suction force applied by the suction unit 325. Moving both the second valve 350 and the first valve 330 into the downward position may be used to irrigate the second connector 316, the first tube 321, and the interiors of the first chamber 312 and the second chamber 313.

As such, the suction and supply unit 300 according to the present invention can perform supply of the irrigation fluid, suction of blood or contaminated fluids from a patient's abdominal cavity, or irrigation of the second connector 316, the first tube 321, and the interiors of the first chamber and the second chamber 313 simply through manipulation of the first valve 330 and the second valve 350, thereby improving user convenience.

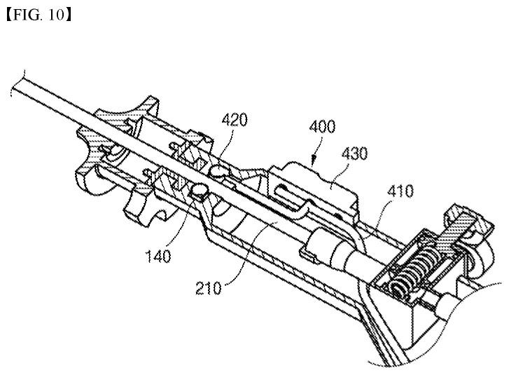

FIG. 10 is a sectional view of the suction and irrigation apparatus of FIG. 1, focused on an electricity power supply unit thereof.

Referring to FIG. 10 in conjunction with FIG. 2 and FIG. 3, the suction and irrigation apparatus may further include an electricity supply unit 400.

The electricity supply unit 400 may have a lead 410, a lead ring 420, and a switch 430.

The lead 410 may be disposed in the mounting space 110 of the handle 100 and may receive external electricity.

The lead ring 420 may be disposed in the second mounting groove 140 and may be connected to the lead 410.

The lead ring 420 may be formed in an annular shape surrounding the outer peripheral surface of the cannula 210 and may remain in close contact with the cannula 210 during rotation of the cannula 210. In this way, the electricity supply unit 400 can stably supply electricity to the cannula 210 during rotation of the cannula 210.

The switch 430 may be disposed on the handle 100 and may be connected to the lead 410 to turn on/off supply of electricity to the lead ring 420.

Although some embodiments have been described herein, it should be understood that these embodiments are provided for illustration only and are not to be construed in any way as limiting the present invention, and that various modifications, changes, alterations, and equivalent embodiments can be made by those skilled in the art without departing from the spirit and scope of the invention. For example, components described as implemented separately may also be implemented in combined form, and vice versa.

The scope of the present invention is indicated by the following claims and all changes or modifications derived from the meaning and scope of the claims and equivalents thereto should be construed as being within the scope of the present invention.

### INDUSTRIAL APPLICABILITY

Laparoscopic surgery uses a surgical instrument for supplying an irrigation solution for irrigation of a patient's surgical site and removing contaminated fluids after irrigation of the patient's surgical site through discharge of the contaminated fluids from the patient's body, that is, a laparoscopic suction and irrigation system.

As the laparoscopic suction and irrigation system, the suction and irrigation apparatus according to the present invention is easy to operate and thus can be widely used in the related industry.

1. A suction and irrigation apparatus comprising:
a handle having a mounting space therein;
an insertion unit extending from the handle and inserted into a patient's abdominal cavity; and
a suction and supply unit disposed in the mounting space and supplying an irrigation fluid to the insertion unit or suctioning blood or other contaminated fluids from the patient's abdominal cavity through the insertion unit,
wherein the suction and supply unit has: a housing having a first chamber and a second chamber separated from each other by an inner partition, a first connector and a second connector connected to the first chamber, and a third connector connected to the second chamber; a first valve provided to the first chamber, allowing a first flow path space to be defined between the first valve and the first chamber, and establishing selective connection between the first connector, the first flow path space, and the second connector while reciprocating in an axial direction of the first chamber; and a second valve provided to the second chamber, allowing a second flow path space to be defined between the second valve and the second chamber, and establishing selective connection between the second flow path space and the third connector while reciprocating in an axial direction of the second chamber, the first chamber and the second chamber being connected to each other through a connection hole formed through the inner partition.

2. The suction and irrigation apparatus according to claim 1, wherein:
the first valve has a first valve body spaced apart from an inner peripheral surface of the first chamber, a first flange protruding from one end of the first valve body and closely contacting the inner peripheral surface of the first chamber, and a second flange protruding from the other end of the first valve body and closely contacting the inner peripheral surface of the first chamber; and
the first flow path space is defined between the first valve body and the inner peripheral surface of the first chamber to be moved in conjunction with movement of the first valve.

3. The suction and irrigation apparatus according to claim 2, wherein the first valve further has a first sealing ring disposed on an outer peripheral surface of the first flange and a second sealing ring disposed on an outer peripheral surface of the second flange.

4. The suction and irrigation apparatus according to claim 2, wherein:
the first valve body has a first insertion groove axially formed at one end thereof; and
the suction and supply unit further has a first resilient member disposed inside the first chamber and having one end supported by the first chamber and the other end inserted into the first insertion groove to resiliently support the first valve.

5. The suction and irrigation apparatus according to claim 4, wherein the first valve body further has a first support protrusion formed on the first insertion groove in the axial direction of the first valve body and disposed inside the first resilient member.

6. The suction and irrigation apparatus according to claim 4, wherein the first chamber has a first securing protrusion surrounding one end of the first resilient member.

7. The suction and irrigation apparatus according to claim 1, wherein the connection hole allows the first flow path space and the second flow path space to be constantly connected to each other regardless of a position of the first valve and the second valve.

8. The suction and irrigation apparatus according to claim 1, wherein the insertion unit has: an electrically conductive cannula rotatably coupled at one end thereof to the first connector, extending from the handle, and coupled to an electrode; an adapter rotatably mounted on the handle and coupled to the cannula to be rotatable in conjunction with rotation of the cannula; an insulating protective tube covering the cannula; and an adjuster mounted on the handle and selectively coupled to the adapter while reciprocating in a longitudinal direction of the cannula.

9. The suction and irrigation apparatus according to claim 8, wherein:
the adapter has a coupling protrusion protruding toward the adjuster;
the adjuster has a coupling groove receiving the coupling protrusion therein; and,
when the adjuster is rotated with the coupling protrusion received in the coupling groove, the adapter is rotated due to friction between the coupling protrusion and the coupling groove.

10. The suction and irrigation apparatus according to claim 8, wherein the insertion unit further has: a clamp coupled to one end of the first connector and having a third through-hole through which the cannula passes; and a sealing member disposed between the clamp and the first connector and allowing the cannula to pass therethrough.
